(19) United States
(12) Patent Application Publication
(10) Pub. No.: US 2011/0178402 A1
(43) Pub. Date: Jul. 21, 2011

(54) ULTRASONIC MEASUREMENT OF pH IN FLUIDS

(75) Inventors: David Hugh Burns, Pointe-Claire (CA); Jonathan Dion, Montreal (CA); Michiko Kato, Montreal (CA)

(73) Assignee: MCGILL UNIVERSITY, Montreal (CA)

(21) Appl. No.: 13/057,567

(22) PCT Filed: Aug. 4, 2009
(86) PCT No.: PCT/CA2009/001064

§ 371 Date: Apr. 1, 2011

Related U.S. Application Data
(60) Provisional application No. 61/085,915, filed on Aug. 4, 2008.
(60) Provisional application No. 61/186,429, filed on Jun. 12, 2009.

(51) Int. Cl. A61B 8/00; C12Q 1/02; G01N 29/02; G01N 33/00; G06F 19/00

(52) U.S. Cl. 600/438; 435/29; 73/61.49; 73/1.02; 702/19

(57) ABSTRACT

The present invention pertains to new methods and apparatuses for the determination of pH of a fluid. Applicants have discovered that ultrasonic spectrophonometry (the study and measurement of acoustic spectra) can be used to distinguish conformational changes of albumin and red blood cells in response to pH. In accordance with the present invention, there is provided a method of determining the pH of a fluid based on a pH-dependent conformation of at least one fluid constituent comprising subjecting the fluid to an ultrasonic pulse; detecting ultrasonic spectral data of the fluid constituent resulting from the ultrasonic pulse; wherein the spectral data varies with pH; and then calculating pH from the spectral data.

### FIELD

The present invention relates generally to ultrasonic measurement of pH in fluids. More specifically, the present invention is related to methods and apparatuses for the determination of hydrogen ion concentration in a fluid based on the pH-dependant conformation of a fluid constituent such as albumin and red blood cells.

### BACKGROUND OF THE INVENTION

Ultrasounds have been used as a diagnostic medical imaging technique for more than 50 years. This is done using the echo of an ultrasound pulse and the echo strength of the pulse to construct an image. The technology is relatively inexpensive and portable. Other well known non-destructive applications of ultrasound are the detection of defects. All acoustic phenomena involve the vibration of particles of a medium moving back and forth.

Human hearing range is typically in the frequency range 20 Hz-20 kHz. Ultrasound is classified as a sound wave with a frequency greater than 20 kHz. Diagnostic medical imaging uses frequencies between 1-10 MHz. The ultrasound wave is non-ionizing radiation which is a mechanical wave and does not have properties like an electromagnetic wave. Therefore, the sound waves need a medium in which they can propagate. The properties of the medium dictate the modes by which ultrasound wave could propagate such as longitudinal, transverse, and surface wave. In a fluid with little or no resistance to shear, only longitudinal waves are propagated. This means that the disturbance will head in the same direction as the propagation of the wave. Ultrasound waves in blood are therefore longitudinal waves.

Savéry and Cloutier teach methods to perform erythrocyte cross-section modeling using high-frequency ultrasound backscattering. Ultimately, they propose to challenge red blood cells (RBCs) with various external stimuli and measure their resonance to diagnose genetic diseases that affect the shape and volume of the red blood cells in response to these challenges.

Based on the previously observed shape and volume change of red blood cells with pH, Gedde et al. set out to identify underlying causes for the rapid and reversible conversion from stomatocyte to echinocyte. They show that red blood cell shape and pH exhibit a non-linear response with a broad pH range in which normal discoid shape is maintained. They also explored membrane potential as an explanation for rapid pH-dependant conformation changes and concluded that membrane potential (from −45 to +45 mV) had no independent effect on red cell shape and did not mediate observed curvature changes. The detection technique used by Gedde et al was not able to detect morphological changes in red blood cells in the pH range 6.4 to 7.9.

The pH-dependent conformation of albumin has been known for some time and albumin's ability to change shape in response to pH and bind multiple blood proteins and drugs has convinced el Kadi et al. to use circular dichroism and ultrasound to study the volume and compressibility of albumin with acidifying pH. Indeed, because albumin has important effects on the pharmacokinetics of various drugs, they wish to study albumin as a model for the development of new tailor-made drug-carriers.

Characterization of pH in complex mixtures has become important in a variety of fields including medical diagnostics, in vitro fertilization, pharmaceuticals, biotechnology, nutraceuticals (functional food), and industrial applications. Since pH is tightly regulated in vivo by many, complex chemical equilibria, one important area of study is the monitoring of pH in bio-fluids such as amniotic fluid, cerebral spinal fluid, culture media and blood as an indicator of functioning metabolism.

For example, blood pH is routinely monitored using external pH electrodes in critical care settings. Regulation of pH between 7-7.8 is critical to patient health. Non-invasive measurement of pH would greatly aid physicians in rapid monitoring and treatment of patients. Likewise, pH is a critical measure of cerebral spinal fluid as an indicator of metabolic disease such as patients with bacterial, tuberculous and fungal meningitis; herpes simplex encephalitis; status epilepticus; and cerebral hypoxia and ischemia. To obtain CSF, lumbar punctures are used which have significant risk and thus, non-invasive measurements are needed. In amniotic fluid, pH of 7-7.5 is considered normal. Variation from this range may indicate abnormal growth or membrane rupture. Non-invasive measurement of pH of amniotic fluid would greatly facilitate clinical diagnosis of these patients. Finally, pH should be tightly controlled in cell culture for any pharmaceutical, stem cell or in vitro fertilization procedure. For these applications there is concern of cross contamination using electrode measurements.

### SUMMARY OF THE INVENTION

Due to the drawbacks of current methods and apparatuses, such as invasive blood draws for measuring blood pH using a pH electrode or indicator strips and due to the absence of non-invasive methods and apparatuses for the determination of pH, it is highly desirable to have a method and apparatus for the non-invasive determination of pH that would be simple to set up and use as well as having fast acquisition time for certain applications in the healthcare industry. This method and apparatus would be useful for non-invasive blood pH determination but also for the pH determination of any fluid containing a constituent with pH-dependent conformation.

The present invention pertains to new methods and apparatuses for the determination of pH of a fluid containing a constituent with pH-dependent conformation such as albumin and/or red blood cells, using statistical analysis of ultrasonic spectral data resulting from ultrasonic probing of the fluid. Applicants have discovered that ultrasonic spectrophonometry (the study and measurement of acoustic spectra) can be used to distinguish conformational changes of albumin and red blood cells in response to pH.

In accordance with the present invention, there is provided a method of determining the pH of a fluid based on a pH-dependent conformation of at least one fluid constituent comprising subjecting the fluid to an ultrasonic pulse; detecting ultrasonic spectral data of the fluid constituent resulting from the ultrasonic pulse; wherein the spectral data varies with pH; and then calculating pH from the spectral data.

In some embodiments of the present invention, calculating pH is performed using linear regression to identify one or more ultrasonic frequencies at which the spectral data varies with pH and the spectral data can be an intensity value as a function of frequency. In this case, the frequency value can be obtained from a time to frequency domain Fourier transform.

In some embodiments of the present invention, the fluid constituent is albumin and/or red blood cells whereas the fluid can be blood, cerebral-spinal fluid, cell culture media and amniotic fluid, urine, lymphatic fluid and intracellular cytoplasm, wherein the fluid can be inside a living person or animal and the ultrasonic pulse is transmitted through a body surface. It will be appreciated by those skilled in the art that any constituent whose conformation varies with pH will allow pH determination using ultrasound spectral data. It will also be appreciated that any fluid which allows the propagation of ultrasound waves can act as the “solvent” for the constituent.

In accordance with the present invention, there is provided a method for determining pH of a fluid using an ultrasound pulse of approximately 5 MHz, the spectral data of the ultrasound pulse being analyzed at frequencies where linear resonance of the constituents affects signal intensity, the frequency range being between 0.5 and 10 MHz.

In accordance with the present invention, there is provided a method for determining the severity of a disease based on the pH of a body fluid which can be indicative of the presence or degree of a disease that affects the pH of a body fluid such as tuberculosis, meningitis, bacterial infections, herpes simplex or fungal encephalitis, metabolic disease (acidosis), status epilepticus, cerebral hypoxia of ischemia.

In accordance with the present invention, there is also provided a method of manufacturing and/or calibrating a device for determining the pH of a bio-fluid comprising identifying a fluid constituent that changes conformation in response to pH; probing the fluid with an ultrasound pulse; detecting ultrasonic spectral data resulting from the pulse; repeating steps the steps of probing and detecting at different pH values of the bio-fluid; identifying frequencies at which intensity varies with pH; and configuring or adjusting the device to detect at least the frequencies.

In accordance with the present invention, there is provided an apparatus for determining the pH of a fluid using ultrasonic pulsing of a fluid constituent whose conformation varies with pH comprising at least one ultrasonic transducer for generating and detecting an ultrasonic pulse; a pulsing device for sending an input signal to the transducer; a detector for receiving an output signal from the transducer; a processor for determining a pH value from the output signal.

In some embodiments of the present invention, the detector comprises one or more narrow band frequency filters. In other embodiments, the time signal is recorded and converted to the frequency domain. Also, the transducer can be either a light-induced, magnetostrictive or piezoelectric transducer.

In accordance with some embodiments of the present invention, there is provided an apparatus for the non-invasive determination of pH wherein all components of the apparatus are comprised in a portable handheld device that can further comprise a pH indicator for indicating the pH of a fluid to a user of the apparatus.

### BRIEF DESCRIPTION OF THE DRAWINGS

Having thus generally described the nature of the invention, reference will now be made to the accompanying drawings, showing by way of illustration, an embodiment or embodiments thereof, and in which:

FIG. 1 is a schematic view of a system for generating vibroacoustic ultrasounds to induce resonance in certain fluid constituents.

FIG. 2 is a schematic view of two ultrasound pH determination instruments. FIG. 2A is one embodiment of an instrument used for calibration purposes and FIG. 2B is one embodiment of an instrument for non-invasive pH determination.

FIG. 3A shows an ultrasonic pulse waveform and a non-linear distortion resulting from the pulse waveform. FIG. 3B shows the spectral broadening resulting from the propagation of a narrow frequency band pulse (in black, centered at 5 MHz) in a fluid due to non-linear distortions.

FIG. 4 shows a typical pH calibration curve for 3 forms of albumin. FIG. 4A is the spectral data for 3 known conformations of albumin and FIG. 4B is the correlation between estimated and measured pH values obtained from the four shown regression frequencies selected (black arrows in FIG. 4A).

FIG. 5 shows a plot of the fractional concentrations of albumin as determined from the literature values of Kf for each form.

FIG. 6 shows a typical pH calibration curve obtained for whole blood.

FIG. 7 depicts the use of spectral ratioing. FIG. 7A is an example of a calibration curve using two concentrations of albumin and spectral ratioing for the calibration. FIG. 7B is a graph of standard deviation as a function of Frequency to show how optimal frequencies can be selected using such a graph.

### DETAILED DESCRIPTION

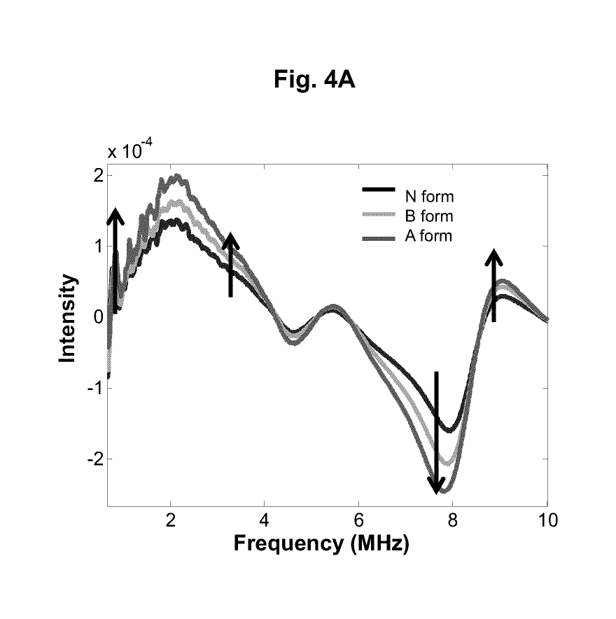

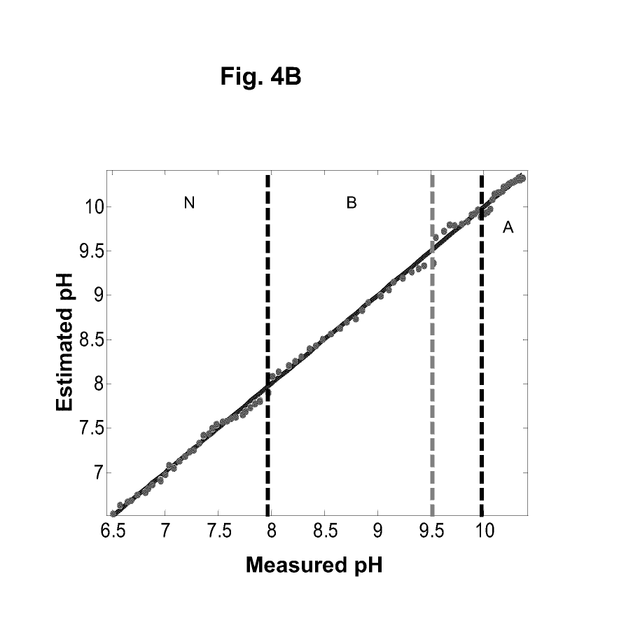

Biological fluids are found over a broad range of pH values, though in human health blood pH values outside of the range 7-7.8 result in death. Many of these fluids contain large quantities of proteins that undergo conformational change in a pH-dependant manner. High frequency sound waves cause certain fluid constituents to resonate at certain frequencies wherein the resonance is affected by the conformation of constituents in the fluid. When ultrasounds propagate through water, they do so non-linearly, i.e. one pulse frequency results in many detectable frequencies (example in FIG. 4B).

Blood can be divided into two components; 55% plasma and 45% cellular components. 92% of plasma is water and other 8% is composed of blood plasma protein, serum albumin, and trace amounts of other materials. 99% of cellular part is the red blood cells (RBCs) and 1% is white blood cells and platelets. Both RBCs and serum albumin are known to undergo the pH-induced shape change. Ultrasonic spectrophonometry was used to observe the response of the blood constituents to the pH deviation from the physiological range.

### Ultrasonic Spectrophonometry System/Instrumentation:

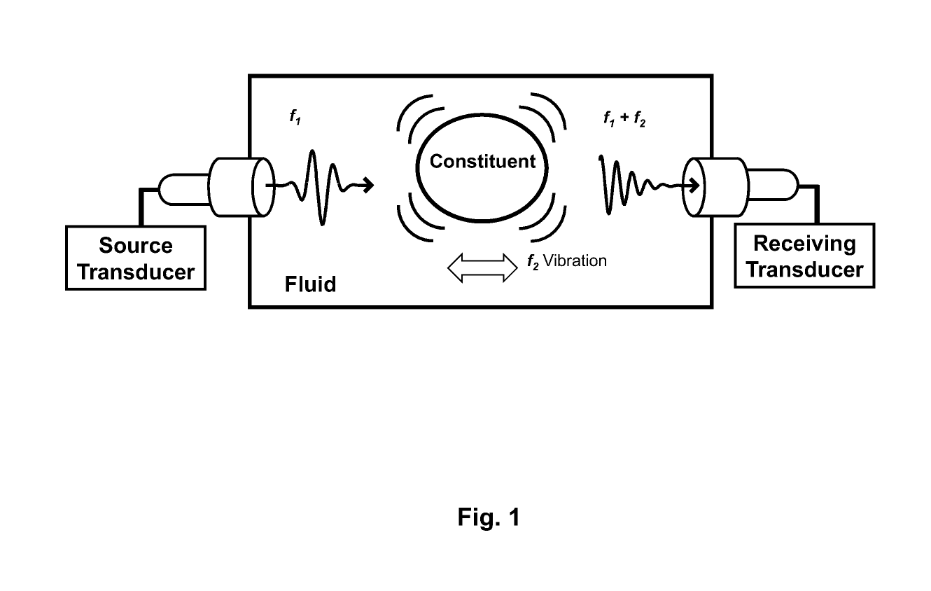

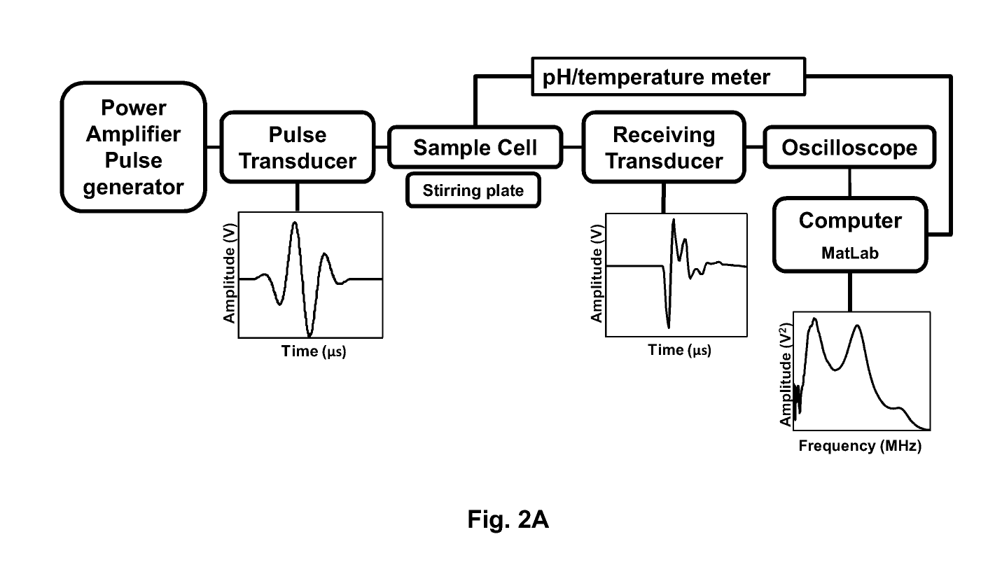

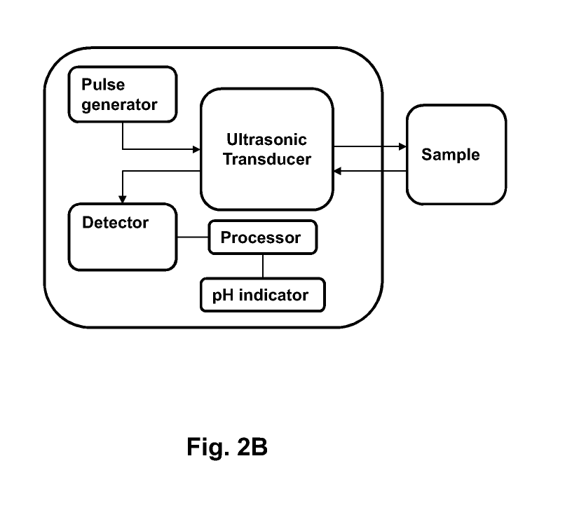

Two transducers with pulsing frequencies of 5 MHz were set up such that they sandwich the sample cell. The two transducers shown are for illustrative purposes because the source and receiving transducers can be the same transducer. A piezoelectric transducer (Russell NDE Systems Inc., Edmonton, Alberta, Canada) is used in the preferred embodiment. The source or probing or pulse transducer, as shown in FIGS. 1 and 2, is understood as meaning the device which converts electrical energy into ultrasound energy for propagating through a fluid. Also, it will be appreciated by those skilled in the art that the spectrophonometry instrument shown in FIG. 2 is used in a research setting for calibrating and determining optimal frequencies and spectral data for unknown fluids and constituents. In such cases, it can be important to have a pH-meter for titration purposes and for establishing correlation between measured pH and estimated pH.

It will be appreciated that, in a device for non-invasive pH determination designed for the healthcare industry, pH and temperature meters, as well as computers and stirring plates are not required (FIG. 2B). Indeed, all calculations can be performed by an electronic circuit or processor that can replace a computer, the electronic circuit or processor being designed to use predetermined frequencies at which pH correlates with intensity of signal for pH calculations. The calculations can add weighted responses at different frequencies according to a multiple linear regression equation developed. Calculations can be electronically or digitally performed. When calculating pH non-invasively, it will be appreciated that using multiple frequencies will help compensate for non-linear effects due to transmission of ultrasonic pulse through a first medium such as a tissue (skin) before reach said fluid.

All components of such an instrument can be designed to fit in a self-contained handheld apparatus for use in the healthcare industry such as hospitals, clinics, emergency rooms, ambulances, etc. It is understood that a self-contained non-invasive instrument for the healthcare industry can be designed to detect only specific predetermined frequencies in order to calculate pH using a predetermined relationship between intensity and pH. Such an apparatus would only require the detection of intensity of signal over time. Specific narrow band filters can be used to capture only these specific predetermined frequencies, or, alternatively, spectral data can be captured and specific frequencies can be obtained from the spectral data. In the latter case, a Fourier transform can be performed to obtain a spectral data plot of intensity as a function of frequency.

Nevertheless, in the apparatus of FIG. 2, sample cells were constructed from a block of plexiglass with acetate sheets as their windows. The pathlength of the sample cell for whole blood was 1.8 cm and holds 1.8 ml of solution. Albumin pH measurements were carried out using a sample cell which holds 40 ml of solution in order to allow for pH titration without significant dilution of albumin solution. Electric pulse from the pulse generator was converted into the ultrasound pulse by one transducer. The transmission pulse was then received at the receiving transducer. The signal was then recorded at the computer through an oscilloscope. The pH, temperature and ultrasonic response of the sample were recorded simultaneously. pH and temperature of the solution inside of the sample cell were measured using Orion Two Star pH meter from Thermo Scientific Inc.

FIG. 1 illustrates general principles of resonance that can apply when generating and detecting vibroacoustic ultrasounds. Vibroacoustic ultrasounds for pH determination:
allow a solution to interact with the ultrasound and linearly resonate or nonlinearly modify propagation of ultrasound depending upon the physical properties of the solution (fluid and pH-dependent constituent);
are conducive for non- to minimally-invasive measurements in highly scattering media such as tissue and blood;
allow for measurement of opaque matrix or fluids;
allow for the development of simple instrumentation with low maintenance that can be used in remote locations by non-expert users;
allow for fast acquisition of spectral data for each sample; and
allow for interpretation of linear and non-linear propagation waves

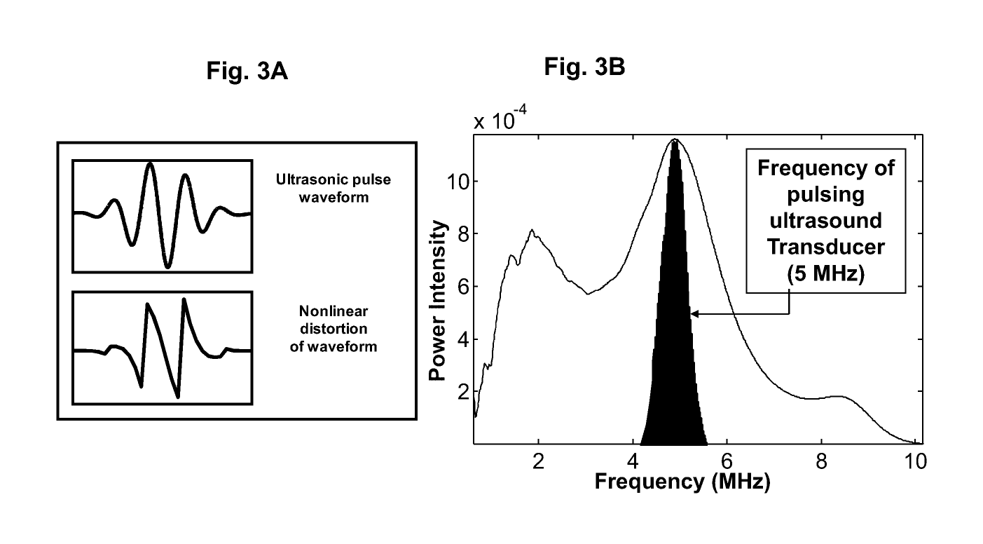

FIG. 3 shows how ultrasonic pulses can propagate as linear and non-linear waveforms. Non-linear distortions which can be caused by various fluid and fluid constituents, can lead to information containing waveforms (FIG. 3A) following analysis of spectral data, including intensity peaks such as those observed around 2 MHz and 8.5 MHz in FIG. 3B.

### Albumin

Serum albumin, a protein of 585 amino acids and molecular weight of 65 kDa, is the most abundant protein of the circulatory system with a concentration between 35 and 50 g/L. Serum albumins contribute to sustain osmotic blood pressure and help in the transport, distribution and metabolism. They are also found in tissues and bodily secretions. Serum albumin undergoes a complex conformal change with pH in aqueous solution. The shape of the albumin becomes elongated towards the two extreme pH ranges. Reversible conformal changes for the pH range between 2.7-10. Albumin is known to exist in structurally-different E (pH<2.7), F (pH 2.7-4.3), N (pH 4.3-8), B (pH 8-10) and A (pH>10) forms (FIG. 4). Bovine albumin was used for some studies but its human albumin counterpart shares 76% homology at the amino-acid level. Human serum albumin has been crystallized and it has been shown that Domain I>domain III>Domain II in terms of susceptibility toward alkaline conformation change.

Transmission ultrasound detection measurements were made in the 0.5-10 MHz frequency range (FIG. 4A). The frequency dependence of the ultrasonic transmissions was characterized for BSA at varying pH values. Distinct spectral signatures were found between the different structural conformations (N-form, B-form and A-form) of albumin (FIG. 4A). The matched water spectrum was subtracted for each form to facilitate optimal frequency identification. To examine the potential of exploiting these spectral changes as a non-invasive measurement of pH, a calibration for pH was determined by step-wise multilinear regression with leave-one-out cross validation. It was possible to prepare a calibration curve for albumin over a wide range of pH values (from 6.5 to 10.5). Discontinuities were observed (see pH 8 and 9.5) in estimated versus measured pH. Interestingly, they correspond with the N to B and the B to A isoform transition points, also coinciding with the known Kf of the albumin conformations (FIG. 4B).

Over a limited pH range (6-8) a calibration was possible using a selected number of frequencies with an R² greater than 0.99 and a SEECV less than 0.05 pH. This is a likely range of pH which would be useful for healthcare industry applications.

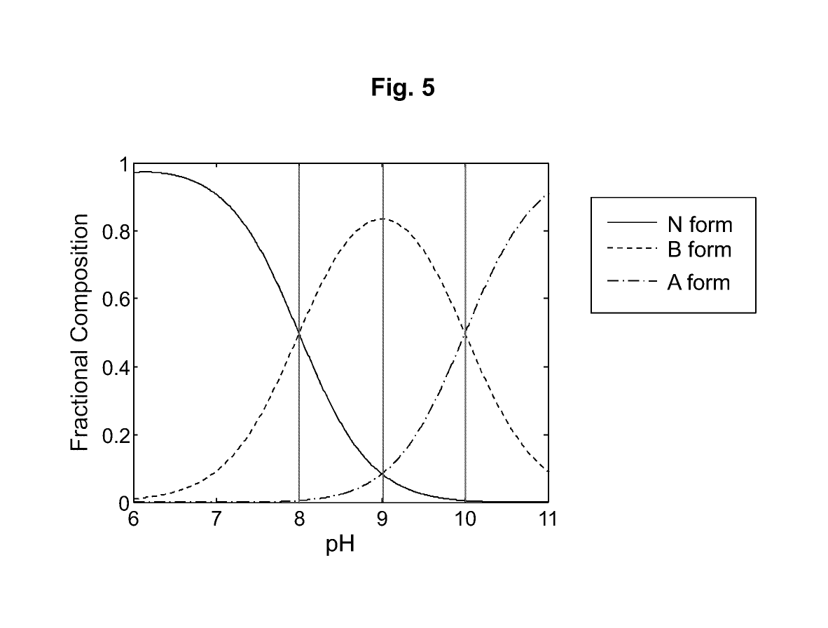

FIG. 5 shows the fractional composition of albumin isoforms as a function of pH, according to the N-form, B-form and A-form categorization. The relative presence of BSA of different forms at the transition pHs may be the reason for the discontinuity in estimated pH observed in FIG. 4B.

TABLE 2
Calibration results for pH-dependent
albumin isoform regression analysis.

| | Full range | N form | B form | A form |
|---|---|---|---|---|
| | (pH 6-11) | (pH 6-8) | (pH 8-10) | (pH 10-11) |
| Regression | 1.22, 3.36 | 1.02, 1.35 | 5.13, 4.03, | 4.42, 1.35, |
| Frequencies (MHz) | | | 9.45 | 5.77 |
| Correlation | 0.99 | 0.99 | 0.99 | 0.96 |
| Coefficient (r²) | | | | |
| Standard Error | 0.08 | 0.05 | 0.02 | 0.05 |
| (SEECV, pH) | | | | |

Temperature deviation ±1.2 C.

Overall, these results offer a convincing demonstration for non-invasive pH determination in biological fluid and media. The results show a strong potential for applications in both research and clinical settings.

### Red Blood Cells

RBCs are the most abundant blood constituent and are known to undergo rapid and reversible shape change as a function of pH and temperature. Stiffness of red blood cells also changes with pH variation (osmotic behaviour). At more acidic pH ranges, they form stomatocytes whereas at more basic pH ranges, they form echinocytes. Scanning electron microscopy images of the RBCs in various pHs has previously been shown and characterized (see Gedde et al. in background). Ultrasound responses from changes in whole blood are influenced by many factors such as hematocrit, plasma viscosity, and red blood cell deformability.

Bovine whole blood was used to demonstrate the possibility of ultrasonic characterization in an opaque, complex sample. Heparinized bovine blood samples were obtained from McGill MacDonald campus and experimentation was performed as soon as possible after receiving the blood samples.

Whole blood was diluted 10× (0.3 ml blood in 2.7 ml PBS) to a range of pH values using phosphate buffered saline (PBS) at specific pH values between 7.0 and 8.0. Monosodium phosphate monohydrate (NaH₂PO₄), disodium phosphate dehydrate (Na₂HPO₄), sodium chloride (NaCl), and potassium chloride (KCl) were dissolved in deionized water to make PBS. A fixed amount of blood was mixed with PBS to achieve the desired pHs. The solution was equilibrated for 5 minutes in altered pH buffers. The measurements were taken in the random order to minimize the instrumental drifts and operator errors and done over a frequency range of 0.5-10 MHz, thus establishing a spectral profile for each sample. The acquisition duration for the spectral profile was one minute. However, the buffer blank measurement and blood sample measurement were alternated in order to measure the blank signals in the similar condition as the sample signals.

Regression analysis of the transmission ultrasound measurements was used to select the characteristic frequencies for the whole blood. Multi-Linear Regression (MLR) with Leave-One-Out Cross-Validation (LOO-CV) was applied on the frequency range. Calibrations on whole blood were possible within this pH range. Calibrations with independent validation were done using a selected number of frequencies with r² greater than 0.95 and SEECV less than 0.07 pH units for whole blood.

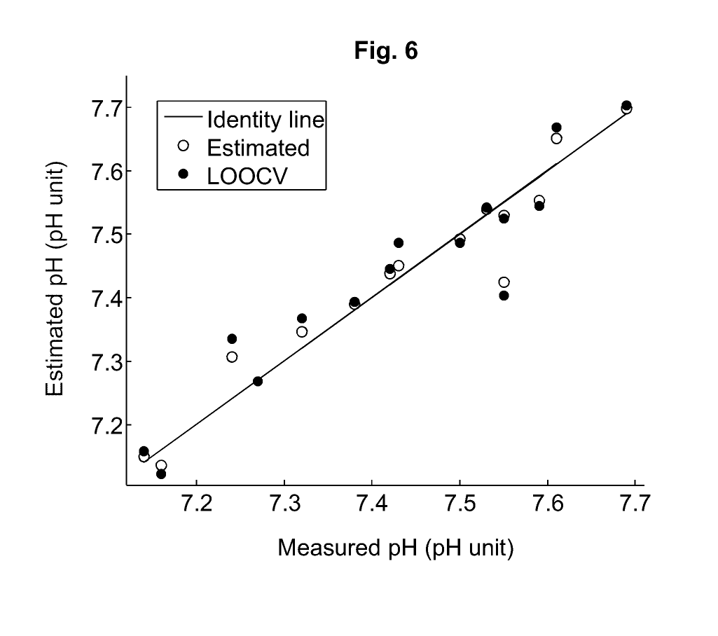

FIG. 6 is the graphic of estimated versus measured pH correlation to show the strong predictive capacity of blood pH using whole blood and the regression analysis shown in Table 3 for the same data. Table 3 shows the frequencies selected for the multiple linear regression (MLR) analysis with leave-one-out cross validation (LOO-CV) performed for whole bovine blood diluted in predetermined amounts of PBS at predetermined pH values. Overall, the results show that pH of bovine blood can be calculated with a precision 0.06 pH units by selecting and using 4 frequencies.

TABLE 3
Calibration results for bovine blood regression analysis.

| | |
|---|---|
| Regression frequencies (MHz) | 9.77 8.46 9.73 9.81 |
| R² (R²CV) | 0.94 (0.89) |
| SEE (SEECV) (pH unit) | 0.04 (0.06) |

Urine is fluid that can be exploited to measure pH changes using ultrasounds. Such changes can be indicative of diseases like metabolic acidosis, and others. It will be appreciated by those skilled in the art that pH measurements can be performed using ultrasound propagation through urine due to the presence, in some circumstances, of albumin and red blood cells in urine. In such circumstances, pH determination can be performed non-invasively in the bladder, in the kidney/nephron, ureter, urethra but also ex vivo in a test tube or flow cell. The presence of albumin and/or red blood cells in the urine increases in certain kidney diseases and can be exacerbated by other diseases such as diabetes, hypertension etc. Furthermore, urine can be a good “reflection” of blood with the added advantage of having many fewer contaminants to facilitate specific spectral profile detection.

Intracellular pH can also be calculated using ultrasound spectral profile analysis. In such situations, the constituent could be a ubiquitous cytoplasmic protein like actin whose conformation is pH-dependent. Indeed, actin demonstrates several favorable characteristics such as being soluble and, when in the unpolymerized state, freely floating in the cytoplasm. It will be understood by those skilled in the art that any intracellular cytoplasmic protein whose conformation is dependent on pH and whose spectral profile allows for pH-intensity correlation analysis can be exploited for pH determination. Although the intracellular volume of one cell is small and would require a very sensitive detection device, specific cell types can be isolated, purified and bathed in a media that does not interfere with intracellular pH determination, therefore allowing for greater sample volumes for spectral profiling.

### Data Analysis:

The albumin and whole blood data were acquired and analyzed using following methods:
Fourier transformation
blank correction (if necessary)
multi-linear regression (MLR)
leave-One-Out Cross-Validation (LOO-CV)
F-test

Data is acquired in the time domain and a discrete Fourier Transform gives the power spectra, which are used in further analyses. Multi-Linear Regression (MLR) allows to correlate changes in frequency response with pH changes. Frequency ranges of highest correlation must be identified and output can be determined by multi-linear model below:

y=m₀+m₁x₁+m₂x₂+ . . . +mₙxₙ

Leave-One-Out Cross-Validation allows applicant's to evaluate the regression model by omitting one sample from data. Estimation is then carried out for the omitted sample and repeated for each sample of data. Finally, an F-test will allow applicants to determine the most parsimonious model.

The above described data analysis methods can be optimized for situations when the constituent concentration (such as albumin) is not precisely known. Indeed, concentration of the constituent may change between individuals and during the course of a long measurement. In these cases, determining the relative concentration changes of the constituent between forms is necessary. Since there are at least three species present in a typical measurement (conformation 1, conformation 2 and background), measurements of unknown concentration of a species can benefit from ratioing of the species to a reference.

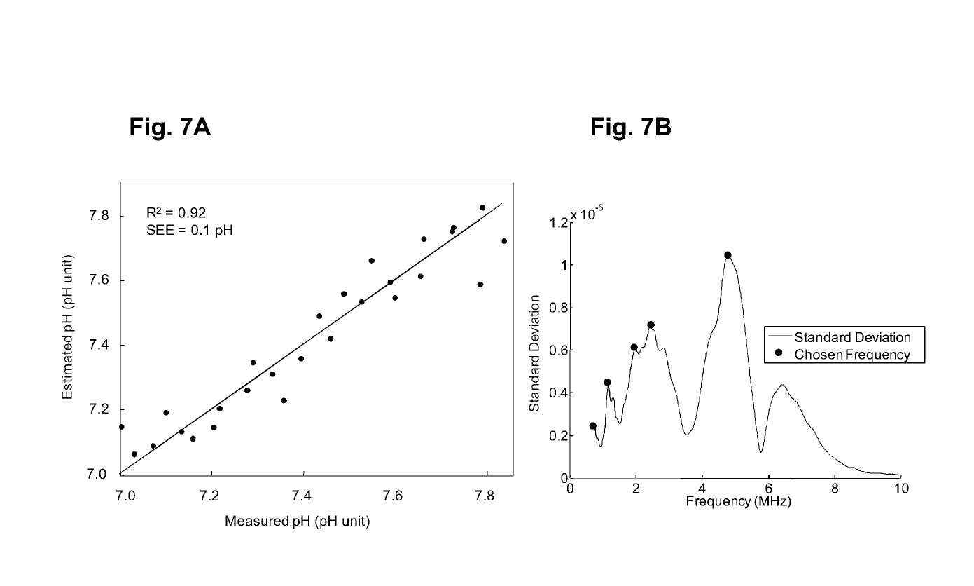

Calibration experiments using mixed concentrations of albumin were performed. FIG. 7A shows the calibration curve using combined data from 40 g/L and 50 g/L of albumin as well as combinations of equations A and B, where B is the formula exploiting the spectral ratioing technique. Local maxima and minima of variations can be included for all possible ratios as well as the multiple linear regression frequencies found in the previous calibrations. The local maxima indicate the maximum variations and the local minima indicate the background or control of the variations. The first equation below (A) uses the linear combination of original frequencies for establishing a formula whereas the second equation (B) uses the linear combination of the ratio of two frequencies. These results demonstrate advantages of using this technique for determining the pH of a solution of unknown and/or varying constituent concentration.

α₁f₁+α₂f₂+ Equation A

αₙfᵢ/fⱼ+αₙ₊₁fₖfₗ+ Equation B

Several approaches for performing spectral ratioing can be used. First, combinations of all possible frequencies can be examined or some optimized search of these frequencies to determine the best combination of frequencies which will reduce variations due to concentration changes in the samples. Many different optimization algorithms can be used such as a genetic algorithm or a Monte Carlo simulation. A convenient method is to look at the signals which have the greatest and least variations in all signals obtained. To facilitate identification and limit the number of these signals, a graph of standard deviation as a function of frequency can be generated (FIG. 7B) and certain points can be identified and used to determine the best combination of all possible ratios with the background signal. Likewise, if additional information is available as to the pH of a test solution, then a correlation can be determined and all possible combinations of these frequencies can be determined.

1. A method of determining the pH of a fluid based on a pH-dependent conformation of at least one fluid constituent comprising:
subjecting said fluid to at least one ultrasonic pulse;
detecting ultrasonic spectral data of said fluid constituent resulting from said ultrasonic pulse; wherein said spectral data varies with pH; and
calculating pH from said spectral data.

2. The method of claim 1 wherein said calculating pH comprises establishing a relationship between spectral data and pH and using said relationship to calculate pH from said spectral data.

3. The method of claim 2 wherein said relationship is established using regression analysis to identify one or more frequencies selected to reduce an error in pH estimation when using said frequencies for calculating pH.

4. The method of claim 1 wherein said spectral data is an intensity value as a function of frequency, said spectral data obtained using a Fourier transform of the time domain.

5. The method of claim 1 wherein said at least one constituent is albumin.

6. The method of claim 1 wherein said at least one constituent is red blood cells.

7. The method of claim 1 wherein said fluid comprises at least one of blood, cerebral-spinal fluid, cell culture media and amniotic fluid.

8. The method of claim 1 wherein said fluid comprises at least one of urine, lymphatic fluid and intracellular cytoplasm.

9. The method of claim 1 wherein said calculating pH comprises compensating for non-linear effects due to transmission of said ultrasonic pulse through a first medium prior to reaching said fluid.

10. The method of claim 1 wherein said ultrasonic probing consists of a pulsing ultrasound frequency of approximately 5 MHz.

11. The method of claim 1 wherein said spectral data is collected for a frequency range between 0.5 and 10 MHz.

12-13. (canceled)

13. The method of claim 1 further comprising determining severity of a disease known to affect pH of a body fluid.

14. The method of claim 13 wherein said disease is comprised in a group consisting of tuberculosis, meningitis, bacterial infections, herpes simplex or fungal encephalitis, metabolic acidosis, status epilepticus, cerebral hypoxia or ischemia.

15. A method of calibrating a device for determining the pH of a bio-fluid comprising:
identifying a fluid constituent that changes conformation in response to pH;
probing said fluid with an ultrasound pulse;
detecting ultrasonic spectral data resulting from said pulse;
repeating the steps of said probing and said detecting at different pH values of said bio-fluid;
identifying frequencies at which intensity varies with pH; and
adjusting said device to detect at least said frequencies.

16-17. (canceled)

18. An apparatus using ultrasound to determine the pH of a fluid containing a constituent whose conformation varies with pH comprising:
at least one ultrasonic transducer for generating and detecting an ultrasonic pulse;
a pulse generator for sending an input signal to said at least one transducer;
a detector circuit connected to said at least one transducer for providing an output signal; and
a processor for determining a pH value using said output signal.

19. The apparatus of claim 18 wherein said output signal is obtained in the time domain and a Fourier transform provides spectral data used by said processor for determining pH.

20. The apparatus of claim 18 wherein the detector circuit comprises one or more narrow band frequency filters.

21. (canceled)

22. The apparatus of claim 18 wherein all components are comprised in a portable hand held device.

23. The apparatus of claim 18 further comprising a pH indicator for indicating pH of a fluid.

24. The apparatus of claim 18 wherein said device is adapted for determining pH non-invasively in a person or animal.

25-26. (canceled)
